(19) United States
(12) Patent Application Publication
(10) Pub. No.: US 2013/0110034 A1
(43) Pub. Date: May 2, 2013

(54) Dialysis Administration Set

(71) Applicant: Cosme Cruz, Chicago, IL (US)

(72) Inventor: Cosme Cruz, Chicago, IL (US)

(21) Appl. No.: 13/478,818

(22) Filed: May 23, 2012

Related U.S. Application Data
(60) Provisional application No. 61/490,200, filed on May 26, 2011.

(51) Int. Cl. A61M 1/28

(52) U.S. Cl. CPC A61M 1/28
USPC 604/29

(57) ABSTRACT

A fixable port for use in dialysis treatment, the port having a base including a first end and a second end, each of the first and second ends having an opening and communicating through a chamber, the base having a ridge to extend into the chamber, the ridge positioned adjacent the base first end; a plunger configured to slidably fit within the base, the plunger having a first end and a second end, the plunger having a first opening and a second opening communicating through a passage, the plunger having a side wall and is configured in cylindrical shape, the plunger wall having the plunger first opening, wherein the plunger first end and the base ridge are shape to cooperate when positioned to abut blocking flow through the base; the plunger having a cowl extending from the plunger second end over the base.

### FIELD OF THE INVENTION

The invention relates generally to devices used in the administration of medicinal fluids into the body. More specifically, the invention relates to devices useful in performing peritoneal dialysis for the treatment of acute and/or chronic renal failure.

### BACKGROUND OF THE INVENTION

The kidneys have an important role in maintaining health. When healthy, the kidneys maintain the body's internal equilibrium of water and dissolved minerals (sodium, potassium, chloride, calcium, phosphorus, magnesium, sulfate). Those acidic end products of metabolism that the body cannot get rid of via respiration are excreted through the kidneys. The kidneys also function as part of the endocrine system metabolizing numerous hormones and producing other hormones such as erythropoietin and calcitriol, among many others. Erythropoietin is involved in the production of red blood cells and calcitriol plays a role in bone formation.

Renal failure is described as a decrease in the glomerular filtration rate. Biochemically, renal failure is typically detected by an elevated serum creatinine level. Problems frequently encountered in kidney malfunction include abnormal fluid levels in the body, deranged acid levels, abnormal levels of potassium, calcium, phosphate, and (in the longer term) anemia as well as cases of loss of mineral (calcium) from bones. Depending on the cause, hematuria (blood loss in the urine) and proteinuria (protein loss in the urine) may occur.

Renal failure can be divided into two major categories: acute kidney injury or chronic kidney disease. The type of renal failure is determined by the rate of which the serum creatinine arises. Other factors which may help differentiate acute kidney injury from chronic kidney disease include anemia and the kidney size on ultrasound. Chronic kidney disease generally is accompanied by anemia and small kidney size.

Symptoms can vary from person to person. Someone in early-stage kidney disease may not feel sick or notice symptoms as they occur. When kidneys fail to filter properly, waste accumulates in the blood and the body, a condition called azotaemia. Very low levels of azotaemia may produce few, if any, symptoms. As the disease progresses, symptoms become noticeable (if the failure is of sufficient degree to cause symptoms). Renal failure, accompanied by noticeable symptoms, is termed uraemia.

Although, reneal replacement treatment by dialysis is able to maintain life it is fraught with many problems and is associated with complications arising by its very nature. Furthermore the endocrine functions of the kidney are not replaced by dialysis.

Dialysis works on the principles of the diffusion of solutes and ultrafiltration of fluid across a semi-permeable membrane. Diffusion describes a property of substances in water. Substances in water tend to move from an area of high concentration to an area of low concentration. Blood flows along one side of a semi-permeable membrane, and a dialysate, or special dialysis fluid, flows along the opposite side. A semipermeable membrane is a thin layer of material that contains various-sized holes, or pores. Smaller solutes and fluid pass through the membrane, but the membrane blocks the passage of larger substances (for example, red and white blood cells, large proteins, large molecular size elements such as Beta 2 micro globulin etc. Forms of these compounds of large molecular size are thought to cause uremic symptoms etc.).

The two main types of dialysis, hemodialysis and peritoneal dialysis, remove wastes and excess water from the blood in different ways. Hemodialysis (HD) removes wastes and water by circulating blood outside the body through an external filter, called a dialyzer, that contains a semipermeable membrane. The blood flows in one direction and the dialysate flows in the opposite direction. The counter-current flow of the blood and dialysate maximizes the concentration gradient of solutes between the blood and dialysate, which helps to remove more urea and creatinine from the blood. The concentrations of solutes (for example potassium, phosphorus, and urea) are undesirably high in the blood, but low or absent in the dialysis solution. Constant replacement of the dialysate ensures that the concentration of undesired solutes is kept low on this side of the membrane. The dialysis solution has levels of minerals such as potassium and calcium that are similar to their natural concentration in healthy blood.

For another solute, bicarbonate, dialysis solution level is set at a slightly higher level than in normal blood, to encourage diffusion of bicarbonate into the blood. The bicarbonate acts as a pH buffer to neutralize the metabolic acidosis that is often present in these patients. The levels of the components of dialysate are typically prescribed by a nephrologist according to the needs of the individual patient.

In peritoneal dialysis (PD), wastes and water are removed from the blood inside the body using the peritoneal membrane of the peritoneum as a natural semipermeable membrane. Wastes and excess water move from the blood, across the peritoneal membrane, and into a special dialysis solution, called dialysate, in the abdominal cavity which solution has a composition similar to the fluid portion of blood.

In peritoneal dialysis, a sterile solution containing glucose is run through a tube into the peritoneal cavity, the abdominal body cavity around the intestine, where the peritoneal membrane acts as a semipermeable membrane. The peritoneal membrane or peritoneum is a layer of tissue containing blood vessels that lines and surrounds the peritoneal, or abdominal, cavity and the internal abdominal organs (stomach, spleen, liver, and intestines). The dialysate is left there for a period of time to absorb waste products, then it is drained out through the tube and discarded. This cycle or “exchange” is normally repeated 3-4 times during the day, (sometimes more often overnight with an automated system). An exchange is each time the dialysate fills and empties from the abdomen. Dwell time means the time the dialysate stays in the patient's abdominal cavity—wastes, chemicals and extra fluid move from the patient's blood to the dialysate across the peritoneum.

The drain process occurs after the dwell time; the dialysate full with waste products and extra fluid is drained out of the patient's abdomen. Ultrafiltration occurs via osmosis; the dialysis solution used contains a high concentration of glucose, and the resulting osmotic pressure causes fluid to move from the blood into the dialysate. As a result, more fluid is drained than was instilled.

Any number of devices are known to those of skill in the art. Previously dialysis sets have been proposed using any number of sealing mechanisms, clamps, connectors and valves. They by and large have been adapted from other applications and are sometimes cumbersome other times inefficient or difficult to operate with high degree of opportunity for touch-contamination all have limited flow capability often with in vivo flow rates of <350 ml/min. Other problems may arise. Dialysis treatment may continue indefinitely. Use and reuse of equipment may result in structural fatigue and compromise. This often requires replacement of equipment at great expense and pain to the patient. Sterility problems can arise given the proximity of the dialysis equipment to the human body.

As a result, there is an ongoing need for devices which address concerns such as device fatigue or failure, sterility and poor flow capability, among others.

### SUMMARY OF THE INVENTION

In accordance with a first aspect of the invention there is provided a dialysis set or fixable port, the port including a base including a first end and a second end, each of the first and second ends being open to form a chamber within the base, the base including a ridge, wherein the ridge is configured to extend into the chamber, a plunger including a first end and a second end, the plunger having a first opening and a second opening to form a passage within the plunger, wherein the plunger slidably fits within the base, and wherein the plunger first end is configured to cooperatively abut the base ridge.

In accordance with a further aspect of the invention there is provided a fixable port for use in dialysis treatment, the port including a base including a first end and a second end, each of the first and second ends having an opening and communicating through a chamber, the base including a ridge to extend into the chamber, the ridge positioned adjacent the base first end, a plunger configured to slidably fit within the base, the plunger including a first end and a second end, the plunger having a first opening and a second opening communicating through a passage, the plunger including a side wall and being configured in cylindrical shape, the plunger side wall having the plunger first opening, wherein the plunger first end and the base ridge are shaped to cooperate when positioned to abut blocking flow through the base, the plunger including a cowl extending from the plunger second end over the base, wherein the plunger side walls include protrusions, the base second end having indentions adjacent the base second end, wherein the plunger protrusions and the base indentations are sized and positioned to cooperate in locking the base and plunger.

In accordance with an even further aspect of the invention there is provided a fixable port, the fixable port including a base including a first end and a second end, the first end having a first opening and the second end having a second opening, the fixable port including a retractable cowl positioned adjacent the first opening.

This invention in concert with a Y Dialysis administration set that has an ID of 3.75 mm ID—results in dialysate flow capabilities of at least about 1000 ml/min in vivo. With a marked reduction in the time required to perform a dialysis exchange simultaneously as the inflow/outflow times decrease, the percentage of dwell time increases dramatically, thus improving the effectiveness of this treatment modality.

### BRIEF DESCRIPTION OF THE FIGURES

FIG. 1 is a side sectional view of a dialysis administration set in accordance with one aspect of the invention.

FIGS. 2A and 2B are partial sectional views of dialysis administration set shown in FIG. 1 showing the dialysis administration set fixed in the open (FIG. 2A) and closed (FIG. 2B) positions.

FIG. 3 is a plan view of the front side of the dialysis administration set shown in FIG. 1.

FIGS. 4A and 4B are a top plan view and side sectional view, (respectively), of a dialysis administration set in accordance with a further aspect of the invention.

FIG. 5 is a side sectional view of a dialysis administration set in accordance with a further alternative aspect of the invention.

### DETAILED DESCRIPTION OF THE PREFERRED EMBODIMENTS

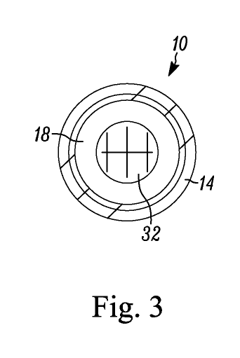

Referring now to the figures wherein various embodiments of the invention are shown through multiple views, with the various elements of the invention identified with like or similar numbers through these views, the invention is a dialysis set or fixable port 10 for use in dialysis treatment. The port comprises a base 12 having a first end 14 and a second end 16, each of the base first and second ends having an opening (18, 20 respectively), and communicating through a chamber 22. The base has a ridge 24 which extends into the chamber 22. As can be seen, the ridge 24 is positioned adjacent said base first end 14. Preferably the ridge 24 is annular or circular following the preferred annular or circular shape of the base first and second openings, FIG. 3.

A plunger 30 is configured to slidably fit within the base 12. The plunger 30 also has a first end 32 and a second end 34. The plunger also has a first opening 36 and a second opening 38 communicating through a passage 39. The plunger has a side wall 40 and is configured in cylindrical shape, FIG. 3. The plunger side wall 40 may have the first opening 36. The plunger first end 32 and the base ridge 24 are shaped to cooperate when positioned to abut by blocking fluid flow through the base 12. The plunger may also comprise a cowl 42 extending from the plunger second end 34 over the outer circumference of the base 12.

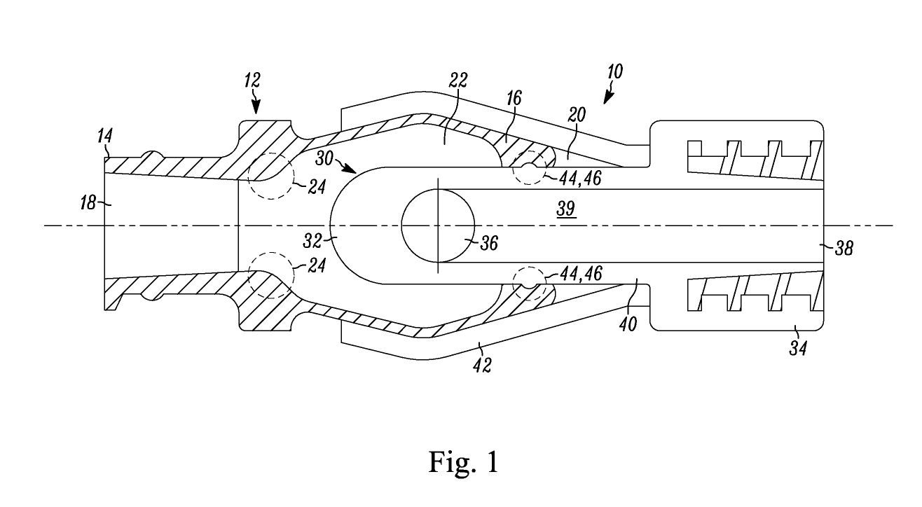

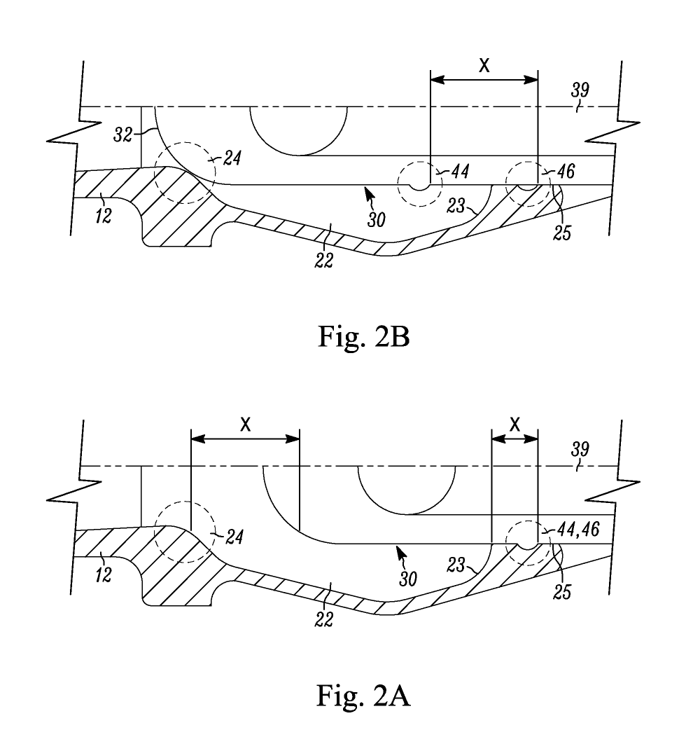

The plunger side wall has protrusions 44, FIGS. 1 and 2A/2B. The base second end 16 has indentions 46 adjacent the base second opening 30. The plunger protrusions 44 and the base indentations 46 are sized and positioned to cooperate in locking or fixing the base 12 and plunger 30 in open and closed positions, FIG. 2A and 2B, respectively.

Generally, the port is fixable in at least two positions as can be seen, in FIG. 2A and 2B. The port is fixable in the open position (FIG. 2A) with the plunger 30 withdrawn allowing flow through a base chamber 22 and the plunger passage 39.

In the second position, FIG. 2B, the plunger 30 is inserted further into the base 12 so that the first end of the plunger rests against the base chamber internal ridge 24 thereby blocking fluid flow through the base chamber 22 and plunger passageway 39.

As can be seen in FIG. 2A, in the first fixed position, the distance between the first end of the plunger and the ridge of the chamber (X) is identical to the distance between the plunger side wall protrusions and the following end of the base 23, (X).

As can be seen in FIG. 2B, when the plunger is fully inserted into the base, the plunger protrusion rests against the base side wall 23 past the point of the base opening wall 25 which otherwise slidably guides the plunger 30 into the base chamber.

Preferably, the plunger and the first and second opening to the base are annular or circular in shape, FIG. 3. The outer circumference of the plunger is slidably guided by the inner circumference of the second opening wall 25 of the base 12, FIG. 3.

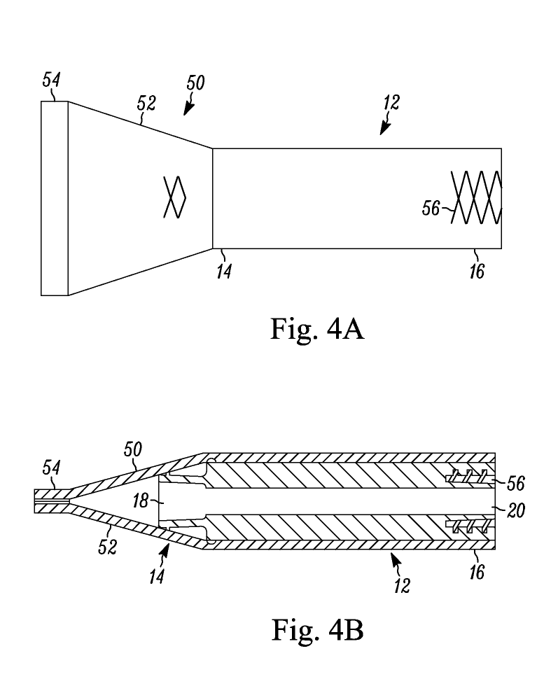

In accordance with a further embodiment of the invention a hooded port is provided, FIGS. 4A and 4B. The dialysis set or fixable port has a base 12 comprising a first end 14 and a second end 16, said first end having a first opening 18 and said second end 16 having a second opening 20, said fixable port comprising a retractable cowl 50 positioned adjacent said first opening 18. The cowl or hood 50 may generally have two portions. The cowl 50 generally covers the port first opening 18 and closes or seals to prevent contamination. Following this function the cowl 50 may be configured through any number of structures.

In accordance with a preferred aspect of the invention, the cowl 50 may comprise two portions. The cowl first portion 52 generally is configured to cover the port first opening 18. The cowl second portion 54 is generally configured to seal the opening 18.

Sealing the cowl second portion 54 may be undertaken through any number of means including the use of structural rubber which is molded to close. The cowl second portion 54 may also be thermally sealed in which case the cowl is opened by cutting or teaming and then closed using structural material which self seals. Another alternative for sealing the cowl 50 is the use of a reclosable seal which may be opened and closed repeatedly during use. As can be seen in the Figures, the cowl 50 may be fastened to the port through means such as a lure lock 56 or complementary threading between the hood and the dialysis set or fixable port.

This retractable cowl 50 is intended to function to cover the end of the dialysis set or port in a way that prevents exposure contamination. As the dialysis set is connected with various lines in preparation in dialysizing the patient, the various locking mechanisms may be exposed by retracting the cowl 56. Once the dialysis set has been connected the cowl may be once again pulled or drawn over the locking or connecting elements. Similarly once the lines are disconnected from the dialysis set the cowl may then be drawn, once again, over the now disconnected exposed locking mechanisms to prevent contamination or the loss of sterility.

Prior to use of the dialysis set, the cowl 50 may also be sealed or closed to maintain or preserve the cleanliness of the dialysis set lock. In use, The seal may either be cut or configured in a way that will allow it to be opened and then reclosed once the dialysis set is taken out of active use. In this fashion, the sterility of the dialysis set may be preserved.

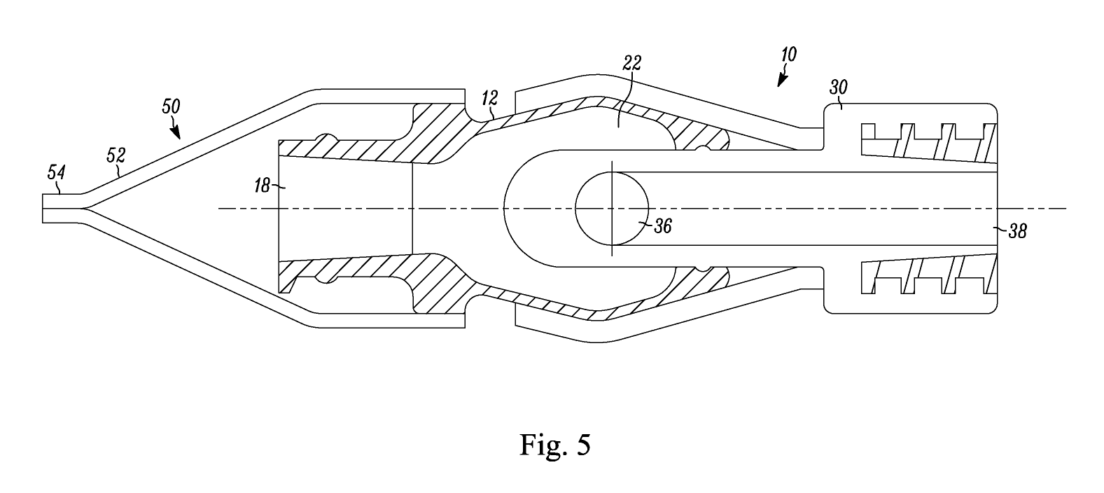

In accordance with a further aspect of the invention, FIG. 5, the cowl 50 may be used to cover the locking portion of the dialysis set earlier disclosed in FIG. 1. Similarly to the embodiment depicted in FIGS. 4A and 4B, the cowl 50 may be opened, retracted and then brought back in place to cover the first end 18 of the base 12.

While the invention has been described above according to its preferred embodiments of the present invention and examples of steps and elements thereof, it may be modified within the spirit and scope of this disclosure. This application is therefore intended to cover any variations, uses, or adaptations of the instant invention using the general principles disclosed herein. Further, this application is intended to cover such departures from the present disclosure as come within the known or customary practice in the art to which this invention pertains and which fall within the limits of the following claims.

1. A fixable port, said port comprising:
a.) a base comprising a first end and a second end, each of said first and second ends being open to form a chamber within said base, said base comprising a ridge, wherein said ridge is configured to extend into the chamber;
b.) a plunger comprising a first end and a second end, said plunger having a first opening and a second opening to form a passage within said plunger;
wherein said plunger slidably fits within said base;
wherein said plunger first end is configured to cooperatively abut said bas ridges.

2. The port of claim 1, wherein said base first and second openings are annular and communicate through an interior chamber, said base comprising a ridge positioned which extends into the base chamber, said ridge positioned adjacent said base first end.

3. The port of claim 2, wherein said base ridge is annular.

4. The port of claim 1, wherein said plunger comprises sidewalls and is configured in a cylindrical shape, said plunger second end having the plunger second opening.

5. The port of claim 4, wherein said plunger side wall has said plunger first opening.

6. The port of claim 5, wherein said plunger first opening is configured adjacent said plunger first end.

7. The port of claim 1, wherein said plunger first end is sized to cooperate with said base ridge.

8. The port of claim 7, wherein said base first end and second end and said base ridge are annular, said base ridge positioned adjacent said base first end, wherein said plunger comprises sidewalls and is configured in cylindrical shape wherein said plunger first end and said base ridge are sized to cooperate.

9. The port of claim 8, wherein when said plunger first end is positioned to abut said base ridge, the base first opening and second opening do not communicate through the chamber.

10. The port of claim 1, wherein said plunger comprises a cowl.

11. The port of claim 10, wherein said plunger cowl extends from said plunger second end over said base.

12. The port of claim 1, wherein said plunger comprises side walls, said plunger side walls comprising protrusions, said base second end having indentions adjacent the base second end, wherein said plunger protrusions and the base indentations are sized and positioned to cooperate in locking the base and plunger.

13. The port of claim 1, wherein said base comprises a lure lock adjacent said base first end and said plunger comprises a lure lock adjacent said plunger second end.

14. The port of claim 1, wherein said base comprises a cowl.

15. The port of claim 1, wherein said base comprises a lure lock adjacent said base first end, said base comprising a retractable cowl adjacent said base first end wherein said cowl covers said lure bock when fully extended.

16. A fixable port for use in dialysis treatment, said port comprising:
a.) a base comprising a first end and a second end, each of said first and second ends having an opening and communicating through a chamber, said base comprising a ridge to extend into the chamber, said ridge positioned adjacent said base first end;
b.) a plunger configured to slidably fit within said base, said plunger comprising a first end and a second end, said plunger having a first opening and a second opening communicating through a passage, said plunger comprising a side wall and is configured in cylindrical shape, said plunger side wall having said plunger first opening, wherein said plunger first end and said base ridge are shape to cooperate when positioned to abut blocking flow through said base; said plunger comprising a cowl extending from said plunger second end over said base,
wherein said plunger side walls comprise protrusions, said base second end having indentions adjacent the base second end, wherein said plunger protrusions and the base indentations are sized and positioned to cooperate in fixing the base and plunger in relative position to each other.

17. The port of claim 16, wherein said base ridge is annular.

18. The port of claim 16, wherein said base comprises a lure lock adjacent said base first end and said plunger comprises a lure lock adjacent said plunger second end.

19. The port of claim 16, wherein said base comprises a cowl.

20. The port of claim 16, wherein said base comprises a lure lock adjacent said base first end, said base comprising a retractable cowl adjacent said base first end wherein said cowl covers said lure bock when fully extended.

21. A fixable port, said fixable port comprising:
a base comprising a first end and a second end, said first end having a first opening and said second end having a second opening, said fixable port comprising a retractable cowl positioned adjacent said first opening.

22. The port of claim 21, wherein said base comprises a lure lock adjacent said base first end and said plunger comprises a lure lock adjacent said plunger second end.

23. The port of claim 21, wherein said base comprises a lure lock adjacent said base first end, said base comprising a retractable cowl adjacent said base first end wherein said cowl covers said lure bock when fully extended.

24. The port of claim 21, wherein said cowl is sealable.
